(19) United States
(12) Patent Application Publication
(10) Pub. No.: US 2022/0049382 A1
(43) Pub. Date: Feb. 17, 2022

(54) METHOD OF MANUFACTURING A COLLAGEN YARN

(71) Applicant: ECCO Sko A/S, Bredebro (DK)

(72) Inventors: Thomas GOGSIG, Bredebro (DK); Stojanka PETRUSIC, Bredebro (DK)

(21) Appl. No.: 17/416,717

(22) PCT Filed: Dec. 20, 2019
(86) PCT No.: PCT/DK2019/050420

§ 371 Date: Jun. 21, 2021

(30) Foreign Application Priority Data
Dec. 21, 2018 (DK) PA 2018 70861

(51) Int. Cl. D02G 3/10; D06B 3/04; A43B 23/07

(52) U.S. Cl. CPC D02G 3/10; D06B 3/04; D10B 2211/06; D10B 2501/043; A43B 23/07

(57) ABSTRACT

A method of manufacturing a collagen yarn includes providing collagen fiber. The collagen fiber is spun into a yarn and tanned subsequent to the spinning of the yarn.

### FIELD OF THE INVENTION

This invention relates to manufactured collagen-based yarn.

### BACKGROUND OF THE INVENTION

Leather is used in a vast variety of applications, including furniture, shoes, clothing, bags, and automobile applications. There is a continuing and increasing demand for leather products and new ways to meet this demand are needed in the view of the economic and environmental cost of producing leather. Further, there is a growing request from users and producers for technological and aesthetic products demonstrating superior strength, uniformity and aesthetic properties that incorporate natural components. A challenge is however that the fiber sources when used displays substandard e.g. in relation to strength and durability and the present invention addresses such needs and interests in an advantageous way.

### SUMMARY OF THE INVENTION

The invention relates to a method of manufacturing a collagen yarn comprising the steps of
providing collagen fiber
spinning the collagen fiber into a yarn and
tanning the yarn subsequent to the spinning of the yarn.

The inventors of the present invention have discovered that by spinning natural collagen fibers into a yarn and tanning the yarn subsequent to the spinning of the yarn several advantages can be obtained, such as relatively high tensile strength, elasticity and regularity.

The described process of tanning may in terms of process flow also be referred to as a post-tanning within the scope of the invention in the sense that the tanning in question is performed after the manufacturing of the collagen-based yarn. Post-tanning in the present context does not necessarily refer to how a skilled person would comprehend the wording that usually applies to e.g. chrome tanning comprising re-tanning, dyeing and fat liquoring or whether the collagen of the yarn has been tanned previously or not. It merely means that the tanning is performed according to the claim-wording, i.e. after the spinning of the yarn.

Unless otherwise stated, the reference to tanning in embodiments or explanation of the invention will refer to the above-described post-tanning, i.e. a tanning which is performed after manufacture of the yarn according to the definition of claim 1.

Advantages of tanning the yarn according to the provisions of the invention e.g. includes is a homogeneous process due to the fact that the material is homogeneous compared to non-woven animal hide. In animal hide, variations in collagen organization are found in animals of different species, areas of the animals and animals of different ages and gender. These variations and differences affect the physical properties of the hides and as a consequence differences in the leather produces from the hides. Collagen organization varies through the thickness of the hide, where the top grain side of the hide is composed of a fine network of collagen fibrils and deeper sections (corium) are composed of larger fiber bundles.

These differences have been minimized according to the provisions of the invention thereby making it possible to apply the right amounts of tanning agents, obtain a faster tanning process when compared to the tanning of leather, obtain a more predictable outcome of the tanning process e.g. in terms of color. It is also attractive that the final product may contain different amounts of the tanning agents and there is a much greater chance that the applied tanning agents is in fact having the desired effect on the collagen.

In the present invention, a fiber may e.g. refer to tightly packed collagen fibrils that forms a fiber and may comprise natural collagen fibers, staple fibers, and filaments and may also comprise additives. The fibers in the present invention may also be shorter than the full length of the yarn or made from one or more continuous strands (filaments) where each component filament runs the whole length of the yarn.

In an embodiment of the invention, the tanning of the yarn is a re-tanning.

The provided collagen-based yarn may, as indicated above, in principle be formed on the basis of non-tanned collagen protein forming part of the yarn. It is however preferred that the performed tanning as not only a post-tanning but also as a re-tanning. This means in practice that the re-tanning is here not only referring to a tanning process which is performed subsequent to the providing of the yarn, but the tanning is also performed on a yarn where collagen protein of the yarn has been subjected to a tanning agent also previous to the manufacturing of the yarn.

The re-tanning is in particular advantageous due to that incorporating fresh chrome into aged wet blue has the effect of creating new cationic sites, which might be useful in fixing anionic reagents later in the process. More fundamentally, the isoelectric point will be moved to a higher value, so that at any pH the charge on the yarn is either less anionic (negative) or more cationic (positive) than the not re-tanned yarn. This too will influence the reactivity towards the tanning reagents, which are often anionic.

Advantages of the re-tanning process in the present invention may be that use of tanning agents may be substantial lower than during the tanning process due to the fact that the fibers may already have been tanned prior to spinning of the yarn. Further, the time spent may also be advantageous lower in the re-tanning process also due to the fact of a possible previous tanning process and due to the open fibrous structure of yarn in comparison with tighter fibrous structure of leather.

The tanning applied within the scope of the invention may e.g. apply to chrome tanning and comprise the processes of tanning, dyeing and fat liquoring. However, in this context it is understood as a tanning process subsequently to the fibers have been manufactured into a yarn. The fibers may or may not have been tanned before manufacturing.

Tanning refers to the conversion of a putrescible organic material into a stable material that resists putrefaction by spoilage bacteria. During the process of tanning, proteins in the fabric are converted into a stable material that will not putrefy while allowing the fabric to remain flexible. The fabric may be stabilized in an “open” form by reacting some of the collagen with complex ions of chromium or other tanning agents.

In embodiments of the invention, fat liquoring may be applied to prevent fiber sticking when the fabric is dried after completion of the wet processes. A secondary effect may be to control the degree of softness conferred to the yarn and/or fabric. One of the consequences of lubrication could be an effect on the strength of the yarn and/or fabric. Fat liquoring may be conducted with self-emulsifying, partially sulfated or sulfonated (sulfited) oils, which might be animal, vegetable, mineral or synthetic. This step might also include processing to confer to the yarn and/or fabric a required degree of water resistance.

In an embodiment of the invention, the collagen fibers are reconstructed fiber staple fibers produced on the basis of a number of mechanically sub-divided or grinded protein fibrils.

In an embodiment of invention, the step of tanning the yarn is performed subsequent to the step of spinning the collagen fiber (CF) into a yarn (Y) and prior to the step of processing the yarn (Y) into a fabric (F).

In an embodiment of invention, the step of tanning the yarn is performed subsequent to the step of spinning the collagen fiber (CF) into a yarn (Y) and subsequent to the step of processing the yarn (Y) into a fabric (F).

In advantageous embodiments of the invention, the two types of tanning methods according to the provisions of the invention may also be combined in the sense that two tanning process steps may be performed, one first tanning of the yarn prior to fabric manufacturing and a further tanning after manufacturing of the fabric.

In an embodiment of invention, the tanning of the yarn includes chemically altering the protein structure of the yarn.

The tanning process of the yarn chemically alters the protein structure by displacing water from the interstices between the protein fibers and cements these fibers together. Tanning increases the spacing between protein chains in collagen from 10 to 17 Å.

In an embodiment of invention, the chemical altering of the protein structure of the yarn includes subjecting the yarn to at least one tanning agent.

Tanning agents may comprise chromium tanning agents including chromium salts like chromium sulfate; vegetable tannins including pyrogallol- or pyrocatechin-based tannins, such as valonea, mimosa, ten, tara, oak, pinewood, sumach, quebracho and chestnut tannins; aldehyde tanning agents including glutaraldehyde and oxazolidine compounds, syntans including aromatic polymers, polyacrylates, polymethacrylates, copolymers of maleic anhydride and styrene, condensation products of formaldehyde with melamine or dicyandiamide, lignins and natural flours; fish oil and other oils.

In an embodiment of invention, the tanning agent comprises acid.

In an embodiment of invention, the tanning agent comprises chromium salts, such as chromium (III) salts.

During chrome tanning chrome alum and chromium(III) sulfate may be used. Chromium(III) sulfate may dissolve to give the hexaaquachromium(III) cation, which at higher pH undergoes processes called olation to give polychromium(III) compounds that are active in tanning, being the cross-linking of the collagen subunits. An outcome of subjecting yarn to chromium is that it looks light blue (known as “wet blue”)

In an embodiment of invention, the tanning agent comprises tannin.

The vegetable tanning process uses raw material/natural tannins, available in liquid or powder form, obtained from different part of plants including woods, barks, fruits, fruit pods, and leaves. The most common tannins are obtained from Chesnut wood, Quebracho wood, Tara pods, Catechu, Chinese gallnut, Turkish gallnut, Gambier, Myrobalan, Oakwook, Sumac, and Valonia Oak.

In an embodiment of invention, the tanning agent comprises syntans.

The term syntan refers to the range of synthetic tanning agents. This class of reagents is wide ranging, but typically they are aromatic, as hydroxy and sulfonate derivatives, so some general principles regarding their functions can be derived.

In an embodiment of invention, the tanning agent comprises combinations for wet white tanning.

Wet white leather is produced by combinations of synthetic tannins, vegetable tannins, glutaraldehydes and mineral, such as aluminum, titanium and zirconium.

In an embodiment of invention, the step of tanning the yarn is preceded and/or includes acidification to the desired pH.

The desired pH will in the present context refer to the pH where penetration of tanning/re-tanning agents into the yarn and/fibers of the yarn are promoted or facilitated.

The pH must be very acidic when the chromium is introduced to ensure that the chromium complexes are small enough to fit in between the fibers and residues of the collagen. Once the desired level of penetration of chrome into the substance is achieved, the pH of the material is raised again to facilitate the process (basification). It is noted that basification in the present context is rather covering the process of raising the pH rather than necessarily to obtain a basic pH end-value. Basification may thus also in the present application be understood as a de-acidification.

The pH may be e.g. lowered (e.g. to pH 2.8-3.2) to allow penetration of the tanning agent and following penetration the pH may be raised to fix the tanning agent (e.g. pH 3.6-4.2 for chrome or higher)

In an embodiment of the invention, the step of tanning the yarn includes fixation of tanning agents at a pH of between 3.6-6.2 without a previous acidification to a pH of between 2.2-3.6.

According to an advantageous embodiment of the invention, the fixation of tanning agent may be performed without previously subjecting the yarn to acidification, as a yarn spun on the basis of collagen fibers under certain circumstances may be made without special consideration to penetration, as it is required in relation to conventional tanning of animal hides.

A further advantage of the invention is that the pH adjusting step is faster than when pH adjusting in the conventional tanning process of leather because the fabric will respond to the acid due to the more accessible conformation of the fabric.

In an embodiment of the invention, the step of tanning of the yarn includes a subsequent de-acidification.

The basification may also be referred to as a neutralization, taking into consideration that the basification is basically understood as an increase in pH after tanning agent(s) has penetrated into the yarn and/or the fibers of the yarn.

The pH must be very acidic when the chromium is introduced to ensure that the chromium complexes are small enough to fit in between the fibers and residues of the collagen. Once the desired level of penetration of chrome into the substance is achieved, the pH of the material is raised again to i.e. fix the tanning agent.

Neutralisation is the process of raising the pH after main tanning, prior to initiating post tanning reactions, to adjust the charge on the leather. The final pH may be below conventional neutrality (pH 7). The mechanism relies on enough basifying power to raise the pH to the required level. Neutralisation and basification are used interchangeably herein.

The pH may be lowered to allow penetration of the tanning agent and following penetration the pH may be raised to fix the tanning agent.

A further advantage of the invention is that the pH adjusting step is faster than when pH adjusting in the conventional tanning process of leather because the fabric will respond to the acid due to the more accessible conformation of the fabric.

In an embodiment of invention, the tanning process comprises temperature adjustment.

A pH adjustment (increase) during a tanning process is normally accompanied by a gradual temperature increase up to 50 degrees Celsius.

In an embodiment of invention, the tanning process comprises a process pressure adjustment.

According to an embodiment of the invention, a part of the tanning concerning tanning agents may be performed at an increased pressure.

In an embodiment of invention, the tanning process comprises at least one drying step.

In an embodiment of invention, the tanning of the yarn includes a mechanically processing the yarn.

During drying process, the fibers may stick together resulting in a stiffer product and mechanical steps may be needed to soften the yarn/fabric.

In an embodiment of invention, the mechanically processing the yarn includes tumbling of the yarn.

In an embodiment of the invention, tumbling is referring to the process of rotating movement in a narrow, high, rotating barrel. The purpose of tumbling is a process of softening the fibers that stick together during drying and possible making the yarn/fabric feel firmer. Shorts rods and shapes in the barrels reinforce the movement of the yarn/fabric.

Stacking also makes the yarn/fabric softer by softening the fibers. A stacker (stacking machine) has two plates with knobs and opposite recesses. Constant vibration ensures the product is punctuated pressed into the recesses by the knobs causing the fibers to stretch. Stacking also reinforces pattern and reduces looseness.

In an embodiment of invention, the tanning of the yarn increases the temperature resistance of the yarn.

In an embodiment of invention, the hydrothermal stability is at least 70 degrees Celsius.

Hydrothermal stability is the measurement of the resistance of a material to wet heat. In the case of collagenic materials, pelt or leather, it is the effect of heat on water saturated material. The shrinkage temperature (Ts) of pelt or leather is the measurement of hydrothermal stability and the principle of method is to suspend the test piece in water, in the form of a strip then to heat the water at a rate of 2 degree Celsius/min and the shrinkage temperature is noted when the sample visible shrinks.

In an embodiment of invention, the tanning process is made at temperatures ranging from about 30 degrees Celsius to about 50 degrees Celsius.

An even further advantage of the invention is that the temperature is lower compared to conventional tanning that are made at temperatures up to 60 degrees Celsius because the fibers in the yarn/fabric are more open and naked compared to leather.

In an embodiment of invention, wherein tanning of yarn is made as a continuedly process by means of rolls pulling the yarn through a tanning compound.

The yarn would possible stick together if processed as fabric and leather and therefore instead treated as a continuedly process on rolls in baths.

In an embodiment of invention, the collagen fiber (CF) is natural collagen fibers.

In an embodiment of invention, the collagen fiber (CF) is reconstructed fibers based on natural collagen.

In an embodiment of invention, the collagen fiber (CF) is a reconstructed fiber based on natural collagen and where the reconstructed fiber comprises at least 40% by weight of natural collagen.

In an embodiment of invention, the collagen yarn comprises at least 40% by weight of collagen protein.

In an advantage of the invention, the collagen yarn may comprise higher amounts of collagen protein by weight and may comprise at least 50% by weight of collagen protein, such as 60% by weight of collagen protein, such as 70% by weight of collagen protein, such as 80% by weight of collagen protein, such as 90% by weight of collagen protein, such as 99% by weight of collagen protein.

In an embodiment of invention, the collagen yarn further comprises non-collagen protein.

In an embodiment of invention, the collagen yarn further comprises 1 to 60% by weight of non-collagen material.

An advantage of the invention is that the collagen yarn may be hybridized with proteins other than collagen with the outcome of a high production flexibility and the possibility to manufacture yarn and fabrics having a high range of different properties.

In an embodiment of invention, the collagen yarn further comprises 1 to 60% by weight of natural and/or synthetic textile fiber.

In an embodiment of the invention, the methods described herein can include an application of a functional ingredient and these ingredients may include, but not limited to a polymer, resin, dye, stain or paint.

In an embodiment of invention, the yarn is post-processed.

Post-processing in the present context is understood as any processes of the yarn comprising but not limited to the process of making fabric including knitting, weaving etc. and further processes such dyeing, impregnating and stabilizing the product.

In an embodiment of invention, the method comprises a step of post-processing the yarn (Y) into a fabric (F).

An advantage of the invention is that the yarn can be processed into fabric that may be used in any way that fabric made from natural fibers is used and may be similar in appearance and feel like leather. A fabric processed from yarn possess the advantages of using different method for processing such as knitting, weaving, crocheting, felting, braiding, plaiting or other methods of textile production known in the art and a fabric is within the invention understood as a cloth or other material made by any of these methods. The manufactured fabric may be similar and used where applicable for textiles and may exhibit properties with diverse strength, flexibility and/or stretch. The fabric may also display a dissimilar property throughout one piece of fabric. By selecting and tuning the content of fibers and manufacturing processing steps, the manufactured fabric can be modified in softness, flexibility, surface texture, elasticity, thickness or other functionalities.

A further advantage of the invention is that the yarn can be processed into fabric with high uniformity and flexibility. Fabric from natural collagen fibers will have significantly less variations in appearance and physical properties compared material produced directly from animal hide.

In an embodiment of invention, the step of post-processing the yarn (Y) into a fabric (F) is carried out by weaving.

Weaving is within the invention understood as a method of textile production in which two distinct sets of yarns or threads are interlaced at right angles to form a fabric.

In an embodiment of invention, the step of post-processing the yarn (Y) into a fabric (F) is carried out by knitting.

Knitting is within the invention understood as a method of textile production by looping one or more continuous yarns.

In an embodiment of invention, the post-processing step comprises ironing.

In an embodiment of invention, the post-processing step comprises dyeing.

In an embodiment of the invention, the dyeing is performed at a pH within 5 to 11, preferably within a pH of 6-8.

In an embodiment of the invention, the dyeing is performed at a pH within 3-8, preferably within 5-6.

In embodiments of the invention, there may be an additional dyeing step and any suitable dye may be used on any type of fabric, despite the background color, although the final effect may be influenced by the previous processes. Applying dye in solution or pigment, to confer dense, opaque color, may be performed in the drum or dyeing agents may be sprayed or spread by hand or machine (padding, roller coating) onto the surface of the leather.

In an embodiment of invention, the post-processing step comprises impregnation.

In an advantageous embodiment of the invention, a tanned and/or re-tanned fabric may be mechanically and/or chemically finished. Mechanically finishing may be used to polish the fabric to yield a shiny or mat surface, iron and plate a fabric to have a flat, smooth surface, emboss a fabric to provide a three-dimensional print or pattern or tumble a fabric to provide a more evident grain and smooth surface. Chemical finishing may involve application of a film, a natural or synthetic coating or other fabric treatment. Fabric may be finished by application of natural or synthetic polymers and/or colorants onto and within the fabric surface to produce a uniform surface, abrasion resistance, water repellency, etc. For instance, acrylic polymers, blends of fluoropolymers and acrylic polymers, and/or silicon polymers of differing molecular weights can be used to impart water repellency and hand feel finish. Finishing may be applied by spraying and/or roller or other applications related to applying finishing. The final properties of the fabric may have incorporated stabilizing and/or lubricating substances during tanning, re-tanning and/or post-tanning.

In an embodiment of invention, manufacturing is with less chemicals than in conventional leather tanning.

An advantage of the invention is that the manufactured fabric may exhibit homogeneousness properties including uniform thickness with uniform distribution of reaction compounds, such as chemicals and/or tanning reagents. The homogeneous properties may result in a lesser use of hazardous chemicals such as chrome and the reuse of chrome tanned hides in the manufacturing process may result in an overall reduced use of chrome and compounds related to the chrome tanning process.

In an embodiment of invention, the manufacturing is performed with chemicals reduced by 10 percent by weight.

In an embodiment of invention, the manufacturing is performed with chemicals reduced by 25 percent by weight.

In an embodiment of invention, the fabric has a tensile strength higher than 10 kPa.

In some embodiments the fabric has a tensile strength of more than 1 MPa or more preferably of more than 10 MPa or even more preferably of more than 50 MPa.

Tensile strength is also known as ultimate tensile strength or tensile testing and is the capacity of a material to withstand loads tending to elongate and break when clamps are attached to the ends of the sample and pulled apart. A fabric may be tested on a textile tensile testing machine.

In an embodiment of invention, the fabric has a shrinkage temperature of more than 70 degree Celsius.

In an embodiment of invention, the fabric has an elastic modulus of more than 100 kPa.

In some embodiments the fabric has an elastic module between 100 kPa and 1000 Mpa.

The elastic module is a number that measures the fabric's resistance to being deformed elastically when applied to a force. The elastic modulus of an object is defined as the slope of its stress-strain curve in the elastic deformation region. A stiff material will have a high elastic modulus and the elastic modulus can be measured using a texture analyzer.

In an embodiment of the invention, the fineness of the fibers of the yarn is less than 10 dTex.

In an embodiment of the invention, the fineness of the yarn is within a range of about 10 Tex to 300 Tex.

In an embodiment of the invention, the content of tanning agent in the collagen fiber is increased during re-tanning of the yarn with at least 10% by weight of the tanning agent contained in the yarn previous to re-tanning.

In a further embodiment, the content of tanning agent in the collagen fiber is increased during re-tanning of the yarn with at least 10% by weight of the tanning agent contained in the yarn previous to re-tanning and where the collagen fiber contains at least 1% by weight of tanning agent which has been provided to the fiber prior to the re-tanning.

In an embodiment of the invention, the yarn after being tanned is fat-liquored and reeled into a plurality of yarn reels. In an embodiment of invention, the yarn is incorporated into an item.

In some embodiments the manufactured yarn will be incorporated into items including but not limited to furniture, clothes, shoes, socks, hand bags, luggage, wallets, jewelry or other consumer, commercial, or industrial products.

In an embodiment of invention, the fabric will be used for an item.

In some embodiments the manufactured fabric will be used for items including but not limited to furniture, clothes, shoes, socks, hand bags, luggage, wallets, jewelry or other consumer, commercial, or industrial products.

In an embodiment of the invention, leather yarn manufactured according to the invention and wherein the yarn is tanned.

In the present context, leather yarn is understood as a yarn at least based on collagen proteins and/or collagen protein fibrils.

An advantage of providing the leather yarn as a tanned material ready for optional further manufacturing is that the final properties of the yarn is substantially known before a fabric or a fabric item is made.

Leather yarn according to the invention, wherein the yarn is wound up on a plurality of yarn reels s prior to dyeing.

An advantage of rolling up the yarn on reels is that the non-dyed yarn then serves as a flexible stock item that can be dyed on order.

In an embodiment of invention, leather yarn according to the invention, wherein the collagen fibers have a tenacity of more than 3 g/denier, such as at least 5 g/denier, such as at least 7 g/denier.

In an embodiment of invention, the amount of tanning agent in leather yarn fabric comprising collagen fiber and tanning agents is less than a tanned leather having the same volume.

In an embodiment of invention, the amount of tanning agent in leather yarn fabric comprising collagen fiber and tanning agents, is less than 10% by volume, such as less than 8% by volume, such as less than 5% by volume, such as less than 2% by volume or such as less than 1% by volume of the fabric.

In an embodiment of invention, the amount of tanning agent in leather yarn fabric comprising collagen fiber and tanning agents, is less than 10% by weight, such as less than 8% by weight, such as less than 5% by weight, such as less than 2% by weight or such as less than 1% by weight of the yarn.

Leather yarn fabric according to the invention and wherein the leather yarn is made according to the invention.

In an embodiment of invention, the amount of tanning agent in leather yarn comprising collagen fiber and tanning agents, is less than 10% by weight, such as less than 8% by weight, such as less than 5% by weight, such as less than 2% by weight or such as less than 1% by weight of the yarn.

Leather yarn fabric according to the invention and wherein the leather yarn is made according to the invention.

In an embodiment of the invention, leather yarn fabric according to the invention, wherein the collagen fibers have a tenacity of more than 3 g/denier, such as at least 5 g/denier, such as at least 7 g/denier.

In an embodiment of the invention, leather yarn comprising collagen fiber and tanning agents, wherein the amount of tanning agent is less than 10% by weight, such as less than 8% by weight, such as less than 5% by weight, such as less than 2% by weight or such as less than 1% by weight of the yarn

In the present context, leather yarn is understood as a yarn at least based on collagen proteins and/or collagen protein fibrils.

In an embodiment of the invention, leather yarn fabric comprising leather yarn according to the invention and wherein the leather yarn is made according to the invention.

In an embodiment of the invention, a leather shoe comprising a sole (SO) and an upper part (UP),

wherein the upper part UP is at least partly comprised of leather fabric,

wherein the leather fabric is produced on the basis of leather yarn according to the invention,

In an embodiment of the invention, the leather shoe according to the invention, wherein the leather shoe comprises the leather fabric according to the invention and wherein the leather fabric is applied in the upper part of the shoe.

In an embodiment of the invention, the leather shoe the leather fabric is applied as a shoe lining SL or as a part of a shoe lining.

The shoe lining is the material inside the shoe that in some applications comes in contact with the entire foot: the sides, top and heels, or at least some of these part of the foot. A purpose of the lining is to cover the inside seams of the shoe and lengthen the shoe's lifespan. Linings made according to the present invention may cushion and comfort the foot or draw out moisture.

Thus, the leather fabric lining feels soft on the skin and will conform to the shape of the foot over time. It's also durable and allows air flow, letting moisture evaporate. Although leather is the highest quality material for lining, certain shoes, particularly athletic shoes, don't use leather lining because the leather adds extra weight. The present invention facilitates such use as the leather fabric may provide all or many of the advantages of conventional leather but also be present in a light weight, thereby increasing to application to e.g. athletic shoes, but also by realizing that the incorporated dye may be matched very well to fabric e.g. in terms of amount contrary to conventional leather where such a process is extremely time consuming and difficult to control in terms of desired end-color.

Overall it is noted that the leather fabric provided according to the present in invention is very breathable and that a shoe provided according to the present invention may not only be relatively color-durable but also be used both as out and optional inner layer of a shoe thereby providing a shoe having an upper part which very breathable compared to conventional shoe fabric.

In an embodiment of the invention, leather yarn according to any of the preceding claims, wherein the yarn comprises tanning agents in at least 1% by weight of the yarn and dye in at least 0.1% by weight of yarn.

In an embodiment of the invention, leather yarn according to any of the preceding claims, wherein the yarn comprises tanning agents in an amount of 1 to 10% by weight of the yarn and dye in an amount of 0.1 to 10% by weight of the yarn in at least 0.1% by weight of yarn.

The invention moreover relates to a fatliquored leather yarn, wherein the leather yarn is tanned and non-dyed.

The invention moreover relates to a fatliquored leather yarn, wherein the leather yarn is non-tanned and non-dyed.

In an embodiment of the invention, the fatliquored leather yarn is manufactured according the invention.

In an embodiment of the invention, fatliquored leather yarn according to the invention, wherein the leather yarn is non-tanned and non-dyed.

Fatliquoring may advantageously be applied to the yarn subsequent of tanning the yarn.

Finishes may also be applied to the method on fibers, collagen yarn and/or collagen fabric. These may include wax surface finishes to improve water resistance, hardening the surface and protect the color. Finishes may include solvents, binders, plasticizers, pigments and specialized waxes.

Further, relevant finishing agents and/or processes suitable for handling yarn textiles may be applied to the method.

The invention moreover relates to a leather fabric manufactured of leather yarn according to the invention.

In an embodiment of the invention, the leather fabric is manufactured of leather yarn, wherein the leather yarn is provided on basis of a solution of protein molecules.

In an embodiment of the invention, the leather fabric is manufactured of leather yarn, wherein the leather yarn is provided on basis of a suspension of protein fibrils.

### THE FIGURES

The invention will be described in the following with reference to the drawings where,

FIGS. 1A and 1B illustrate an example of collagen fibers (CF) twisted and spun to form a yarn (Y) prior to a tanning process (TP),

FIGS. 2A and 2B illustrate an example of collagen fibers (CF) twisted and spun to form a yarn (Y) and cord (CO) prior to a tanning process (TP),

FIG. 3 illustrates the manufacturing of a fabric according to an embodiment of the invention,

FIG. 4 illustrates an example of processing collagen fibers (CF) and (Y) by tanning according to an embodiment of the invention,

FIG. 5 illustrates a shoe according to an embodiment of the invention,

FIG. 6A-D illustrates different tanning strategies within the scope of the invention and where

FIG. 7 illustrates a further advantageous embodiment of the invention.

### DETAILED DESCRIPTION

In the description and claims, the percentage values relating to an amount of material are percentages by weight (wt. %) based on the total weight of the filament or the staple fiber in question unless otherwise indicated. The word “comprising” may be used as an open term, but it also includes the closed term “consisting of”.

As used herein, the singular forms “a”, “an” and “the” include plural referents unless the context clearly dictates otherwise.

As used herein, “at least one” is intended to mean one or more, i.e. 1, 2, 3, 4, 5, 6, 7, 8, 9, 10, etc.

The term “collagen” refers to any one of the known collagen types of the at least 28 distinct collagen types, each serving different functions in animals. The major component of skin is type I collagen although other types of collagen may also be used to form leather. Collagens are proteins, i.e. they are made up of amino acids. They can be separated into alpha-amino acids and beta-amino acids. Each one features a terminal amino group and a terminal carboxyl group, which become involved in the peptide link and a sidechain attached to the methylene group in the center of the molecule. When the amino acids are linked together to form proteins, they create an axis or ‘backbone’ to the polymer, from which the sidechains extend. It is the content and distribution of the sidechains that determine most of the properties of any protein.

The term “collagen fibrils” refers to nanofibers composed of triple helices of collagen molecules (tropocollagen). The fibrils may have diameters ranging from 10-100 nanometers and are usually not found alone but rather a part of greater hierarchical structures. Each fibril may be interlocked with its neighboring fibrils to form a collagen fiber. Collagen fibrils may originate from shavings, leather pulp and/or tanned hides including the waste material from tanning process, non-finished and finished leather scraps. The collagen fibrils may originate from different sources but typically skin.

The term “collagen fiber” refers to tightly packed collagen fibrils that exhibit a high degree of alignment in the direction of the fiber and is often longer than it is wide. Variations of organization or crosslinking of fibrils and fibers may provide strength to the material. Collagen fiber may comprise staple fibers or filaments. The fiber may be a reconstructed fiber and may comprise other material than collagen.

The term “staple fiber” refers to fibers of discrete length and may be of any composition. Staple fiber may e.g. be provided by division of a collagen-based filament into discrete staple fibers of a certain length. The length may vary dependent on the application. In the present context, the length should be chosen to allow subsequent spinning of the staple fibers into a yarn. The staple fibers may be further processes to provide an item, such as a yarn or non-woven material.

The term “reconstructed” fiber refers to staple fibers produced on the basis of a number of mechanically sub-divided protein fibrils. The reconstructed fiber may be formed from a protein suspension directed through a nozzle onto a surface for drying. The suspension is dried to remove water and solvent from the suspension and thereby the reconstrued fiber is formed e.g. on a belt or a cylinder surface.

Such a manufacturing method of reconstructing fibers on the basis of protein fibrils is e.g. illustrated in WO 2018/149950 or WO 2018/149949 for the use in connection with collagen.

Other starting materials may of course be added to the collagen, there facilitating reconstructed fibers based on a mixture of fibrils derived from collagen material such as leather and fibrils derived from e.g. cellulose sources.

The term “wool” refers to a fluffy material of staple fibers or fibers or reconstructed natural based fiber. The term wool may also be used as natural fiber based wool or staple fiber wool.

The term “manufacturing process” as used herein, may be the process where fibers forms an inhomogeneous network of natural fiber based raw wool and processes through carding preparations and carding, where carding includes fiber cleaning, opening, mass reduction, mixing, homogenization and structuring that disentangles and intermixes fibers to produce a continuous web or sliver suitable for subsequent processing such as spinning.

The term “sliver” as used herein, is a long bundle of fiber or rope-like strand of fibers that is generally used to spin yarn and created by carding or combining the fibers, which is then drawn into long strips where the fibers are parallel. A sliver may be slightly twisted and considered as a loose and soft robe-like textile fiber.

Spinning is the twisting together of drawn-out strands of fibers to form yarn and can be processed with ring spinning or other systems could include air-jet, open-end and vortex spinning. Ring spinning is the most common method of spinning fibers to make a yarn by a continuous process where the roving is first attenuated by using drawing rollers, then spun and wound around a rotating spindle which in its turn is contained within an independently rotating ring flyer. The term roving refers to a long and narrow bundle of fiber and are produced during the process of making spun yarn from fibers. Their main use is as fibers prepared for spinning, but they may also be used for specialized kinds of knitting or other manufacturing processes.

Yarn is a long continuous length of interlocked fibers suitable for use in the production of fabric. Yarn may be spun from a variety of materials, including variants of collagen fiber, but the yarn may also include amounts of natural fibers and synthetic fibers within the scope of the invention. The direction in which the yarn is spun is called twist and is characterized as S-twist or Z-twist according to the direction of spinning. Tightness of twist is measured in TPI (twists per inch or turns per inch). Two or more spun yarns may be twisted together or plied to form a thicker yarn. The term plies refer to a process used to create strong balanced yarn and is done by taking two or more strands of yarn that each have a twist to them and putting them together. Filament yarn may be long continuous fibers twisted or grouped together. Multiple filament yarns may be combined into a yarn with different textures. Spun yarn may contain a single type of fiber or be a blend of various types of yarn and possible combining yarn of natural fibers with yarn of synthetic fibers.

The yarn may further be used for a manufactured fabric by weaving, a method of material production where two distinct sets of yarns are interlaced at right angles to form a fabric. The longitudinal yarns are called the warp and the lateral yarns are the weft or filling. The method in which these threads are inter-woven affects the characteristics of the fabric and the three basic weaves are plain, twill and satin but can also be special weaves such as pile, Jacquard, dobby and leno that requires special loom attachments for their construction.

The yarn may also be used for a manufactured fabric by knitting, a method that creates multiple loops of yarn called stitches in a line or tube. Knitting has multiple active stitches and knitted fabric consists of a number of consecutive rows of intermeshing of loops. Different types of yarn (fiber type, texture and twist), needle sizes, and stitch types may be used to active knitted fabrics with different properties such as texture, weight, look, heat retention, color, water resistance, integrity, conductivity and other possible properties.

The yarn may also be subjected to any other manufacturing processes including, but not excluded to, crocheting, braiding and/or knotting.

Tanning is used as the conventional ways of treating leather and may be applied to the invention. Depending on the compounds, the color and texture of the fabric may change. The technical definition of tanning is well known in the art, but briefly, according to Anthony D. Covington “Tanning Chemistry” chapter 10, the only strict definition of tanning is the conversion of a putrescible organic material into a stable material capable of resisting biochemical attack. Tanning involves a number of steps and reactions depending on the initial material and the final product.

In the case of collagen, it is the sidechains that largely define its reactivity and its ability to be modified by the stabilizing reactions of tanning when leather is made. In addition, the chemistry of the backbone, defined by the peptide links, offers different reaction sites that can be exploited in some tanning processes. During the tanning process, modification of collagen by the chemistry of the tanning agent(s) affects the different features of the properties of the material; The hydrophilic-hydrophobic balance of the leather may be markedly affected by the chemistry of the tanning agent by changing the relationship between the leather and the solvent, which in turn could affect the equilibrium of any reagent between the solvent and the substrate. Also, the site of reaction between the reagent and the collagen may affect the isoelectric point of the collagen and consequently there could be a different relationship between pH and charge on the leather. The lower the isoelectric point, the more anionic or less cationic the charge on the pelt may be at any pH value: the higher the isoelectric point, the more cationic or less anionic the charge on the pelt will be at any pH value. Further, the relative reactions at the sidechains and the backbone of the protein could possible determine the type of reaction and hence the degree of stability of the tannage: the fastness of the reagent may be influenced by the interaction between reagents and the substrate.

Hydrothermal stability as used herein could possibly be measured through the shrinkage temperature (Ts) of a hide. This is the temperature at which the energy input (heat) exceeds the energy bound in existing hydrogen bonding of the collagen structure resulting in the decomposition of the helical structure. The shrinkage temperature for untanned hides is generally around 65 degrees Celsius. The Ts may be increased through the process of tanning.

Decitex (dtex) as used herein is the unit of the linear density of a continuous filament or yarn, equal to 1/10 of a tex.

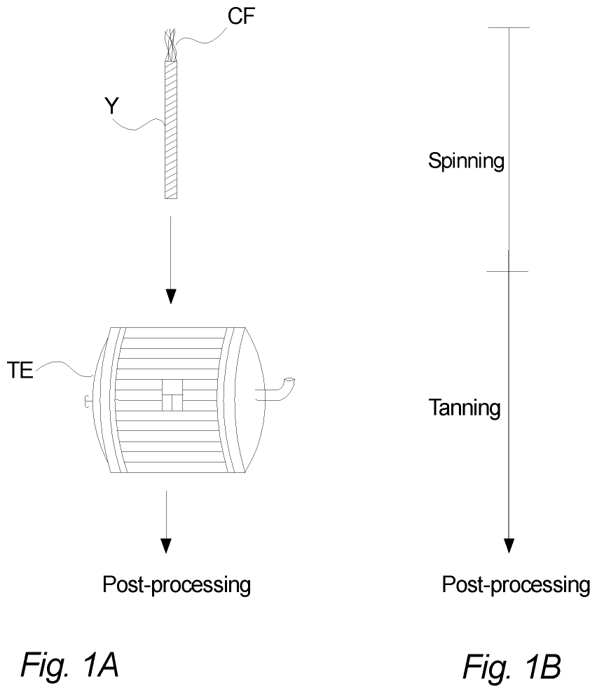

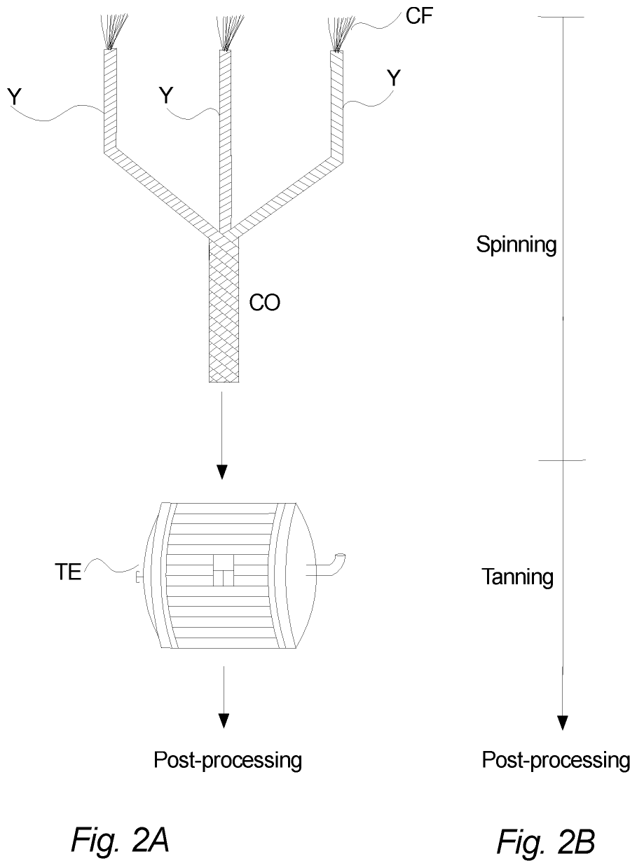

Referring to FIGS. 1A, 1B and 2A, 2B, a schematic view of a process according to an embodiment of the invention is shown.

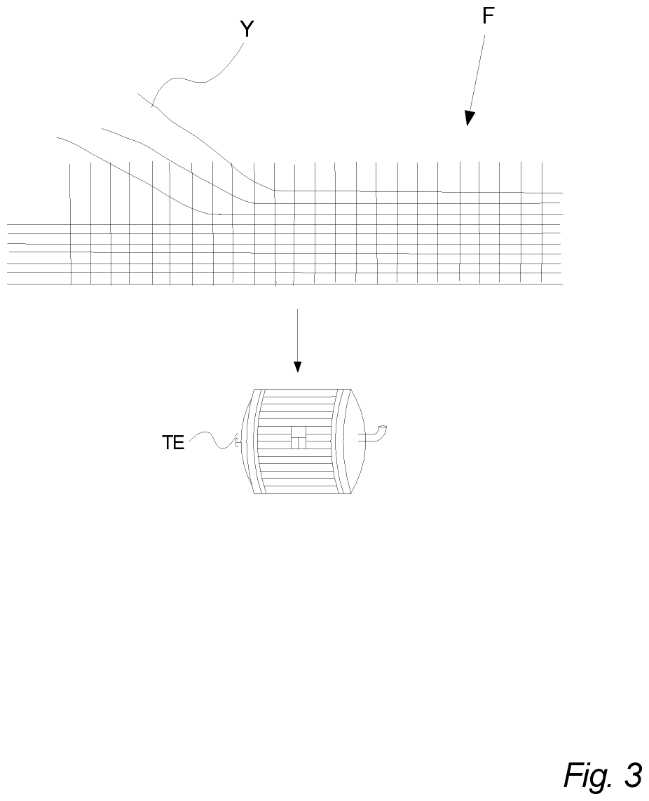

Further embodiments are illustrated in FIGS. 3-6, and all of these embodiments may be understood in the light of FIG. 1-2 and the discussion thereof below.

Returning to FIGS. 1A and 1B illustrating some principles of advantageous embodiment of the invention, FIG. 1A illustrates the mechanical components throughout a corresponding process flow as shown on a process flow time line on FIG. 1B, illustrates as process steps spinning, tanning and post-processing. Initially collagen fibers (CF) in bundles as slivers are spun into yarns (Y) and then subjected to tanning and subsequently to post-processing.

Applicable collagen fiber materials applicable as a basis for the inventive yarn, fabrics and end-items thereof include yarn based on collagen fibers made on the basis of e.g. natural collagen fibers, staple fibers of such or staple fibers of reconstructed filaments or reconstructed filaments, biologically grown collagen fibers and synthesized collagen fibers.

Examples of applicable natural collagen fibers may e.g. be provided according to the loosening method described in EP 1 736 577B or alternatively be manufactured as fibers according to e.g. WO 2018/149950, WO 2018/149949 or U.S. Pat. No. 3,556,969, or WO 2017/142896 or 2007/225631 A1. According to the latter, the collagen fiber should be loosened from the provided non-woven-structure.

The collagen fiber in the present context is generally understood as a fiber comprising collagen protein in an amount of at least 20% by weight of the fiber, such as at least 40% by weight of the fiber, such as at least 60% by weight of the fiber, such as at least 80% by weight of the fiber.

The collagen fiber in the present context has several benefits for use in the present invention, e.g. by providing stretch to a final fabric made according to the provisions of the invention.

Yarn is here understood as a kind of minimum structure of bundled collagen fibers extending in the direction of length. The extension of the bundled structure can in principle be infinite, although this is of course not a real-world application.

In the present embodiment of FIG. 1A and FIG. 1B, relatively short staple fibers of collagen-based fibers, i.e. staple fibers comprising collagen protein, are applied, thereby making it advantageous to apply spinning for the manufacturing of a lengthwise more robust fiber structure. It should nevertheless be emphasized that the present embodiment may be manufactured in the basis of collagen fibers in the form of filaments, whereas the embodiment of FIG. 2A, 2B is more founded on the use of staple fibers.

The spinning process is the twisting together of drawn-out strands or slivers of fibers to form yarn and can be processed with ring spinning or other systems could include air-jet, open-end and vortex spinning. Ring spinning is the method of spinning fibers to make a yarn by a continuous process where the roving is first attenuated by using drawing rollers, then spun and wound around a rotating spindle which in its turn is contained within an independently rotating ring flyer. The direction in which the yarn may be as S-twist or Z-twist according to the direction of spinning and spun yarn may contain a single type of fiber or be a blend of various types of yarn and possible combining yarn of natural fibers with yarn of synthetic fibers.

The tanning (TE) equipment in the present context is provided to perform at least a chemical modification of the collagen protein of the yarn (Y). The tanning process as such is well-known to the skilled person and may be performed in numerous ways as long as a wet-tanning process step is involved and where a chemical reaction is obtained between a tanning agent and the collagen protein of the yarn.

The illustrated tanning step may in principle cover a tanning process design and executed on a yarn, where the collagen proteins of the cord has not previously been tanned. It is a however preferred to regard the tanning step as a re-tanning step in the sense that the collagen fiber subjected to the tanning step has previously be subjected to some kind of tanning agent.

It is however noted that the preferred tanning operation is best described as a so-called post-tanning, involving at least one wet processing step involving a single chemical process or a combination of chemical processes.

Post-processing may be any processes of the yarn comprising but not limited to the process of making fabric including knitting, weaving etc. and further processes such dyeing, impregnating, functionalizing and stabilizing the product.

It should be noted that the illustrated embodiment of FIGS. 1A and 1B is exemplary. Other ways may be applied for the providing a yarn which is suitable for post-processing e.g. provided by weaving or knitting of the provided yarn.

Yarn is here understood as a kind of minimum structure of bundled collagen fibers extending in the direction of length. The extension of the bundled structure can in principle be infinite, although this is of course not a real-world application.

Referring to FIGS. 2A and 2B, the mechanical components throughout a corresponding process flow as shown on a process flow time line on FIG. 2B, illustrates as process steps spinning, tanning and postprocessing. Initially collagen fibers (CF) in bundles as slivers are spun into yarns (Y) and then two or more spun yarns may be twisted together or plied to form a thicker yarn or cord (CO). The term plies refer to process used to create strong balanced yarn and is done by taking two or more strands of yarn that each have a twist to them and putting them together, the strands are twisted together in the direction opposite that in which they were spun.

Spun yarn may contain a single type of fiber or be a blend of various types of yarn and possible combining yarn of natural fibers with yarn of synthetic fibers.

In the present embodiment of FIG. 2A and FIG. 2B, relatively short collagen fibers, i.e. staple fibers comprising collagen protein, are applied, thereby making it advantageous to apply spinning for the manufacturing of a lengthwise more robust fiber structure, here referred to as a cord (CO), although the cord (CO) may also be understood within the prior art and according to the present invention as a yarn, a strengthened yarn.

After the cord (CO) has been provided, the cord is processed with tanning equipment (TE) in a tanning step.

The tanning (TE) equipment in the present context is provided to perform at least a chemical modification of the collagen protein of the cord (CO). The tanning process as such is well-known to the skilled person and may be performed in numerous ways as long as a wet-tanning process step is involved and where a chemical reaction is obtained between a tanning agent and the collagen protein of the yarn/cord.

The illustrated tanning step may in principle cover a tanning process design and executed on a cord/yarn, where the collagen proteins of the yarn/cord has not previously been tanned. It is a however preferred to regard the tanning step as a re-tanning step in the sense that the collagen fiber subjected to the tanning step has previously be subjected to some kind of tanning agent.

It is however noted that the preferred tanning operation is best described as a so-called post-tanning, e.g. involving at least one wet processing step involving a single chemical process or a combination of chemical processes.

Post-processing may be any processes of the yarn or cord (CO) comprising but not limited to the process of making fabric including knitting, weaving etc. and further processes such dyeing, impregnating, functionalizing and stabilizing the product.

It should be noted that the illustrated embodiment of FIGS. 2A and 2B is exemplary. Other ways may be applied for the providing a yarn and cord which is suitable for post-processing e.g. provided by weaving or knitting of the provided yarn. Thus, the difference between FIG. 2A, 2B and FIG. 1A, 1B is basically the way that the final rope-like structure is established; as a yarn (Y) or as a cord (C).

In other words, the inventive post-tanning may be performed on e.g. a yarn or a further processed yarn, such as a cord, or e.g. on a fabric.

Finally, the yarn or cord may typically be post-processed into a fabric (F) and referring to FIG. 3, a fabric processed from yarn or cord may possess the advantages of using different method for processing. The present figures suggest a woven structure, but it should be noted that the figure is also intended to illustrate methods such as knitting, crocheting, felting, braiding, plaiting or other methods of textile production known in the art.

The fabric (F) within the scope of the invention may comprise different fibers and/or yarns such as but limited to wool, cellulose, polyester, silver, elastic etc.

The fabric may be tanned subsequently to the ended process of making the fabric or it may not be subjected to a further tanning process if the yarn has been post-tanned before the manufacture of the fabric, but after manufacture of the yarn

It should be noted that the illustrated embodiment of FIG. 3 is exemplary. Other ways may be applied for the providing a fabric.

It should also generally be noted that the term post-tanning when applied, may be referring to a conventional understanding of post-tanning, including a re-tanning, i.e. a tanning process within the understanding of conventional re-tanning in terms of the applied chemical products, process temperatures, process pH values, etc. In other words, the inventive tanning process would thus typically within the scope of the invention involve at least a conventional re-tanning applied to the yarn or fabric in question if the collagen fiber has been somewhat chemically modified in an earlier tanning process. It should here be noted that “conventional” in no way indicates that re-tanning has previously be applied to yarn according to the provisions of the invention. It merely means that the skilled person may find relevant guidance in conventional re-tanning processes when designing the inventive tanning process.

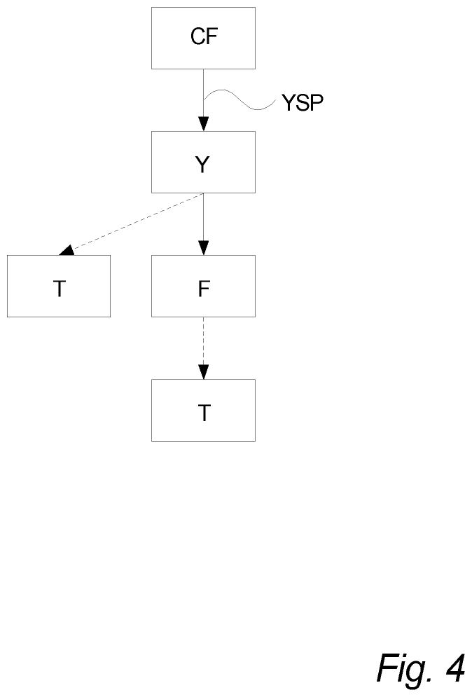

FIG. 4 illustrate a further embodiment of the invention. The embodiment relates to the processing of collagen fibers (CF) into, yarn (Y) and tanned fabric (TF) or the processing of collagen fibers (CF) into yarn (Y) and tanned yarn (TY).

According to the illustrated process flow collagen fibers (CF) are spun into a yarn (Y) by a yarn spinning process (YSP). After this, the yarn (Y) may either be tanned, typically in a re-tanning/tanning process (TP) into a tanned yarn (TY).

Alternatively, the produced yarn (Y) may instead by processed into a fabric (F) based on the collagen yarn (Y) in a fabric manufacturing process (FMP) subsequently be processed in a tanning process (TP) into a tanned fabric (TF).

Technically, it would also be possible to do tanning after both the provision if the yarn (Y) and then add a further tanning step (not shown) when the already tanned yarn (TY) has been processing into a fabric in a fabric manufacturing process (not shown). This may be possible, but not necessarily desirable, as one tanning step should typically enough.

The spinning process (YSP) is the twisting together of drawn-out strands or slivers of fibers to form yarn and can be processed with ring spinning or other systems could include air-jet, open-end and vortex spinning. Ring spinning is the method of spinning fibers to make a yarn by a continuous process where the roving is first attenuated by using drawing rollers, then spun and wound around a rotating spindle which in its turn is contained within an independently rotating ring flyer. The direction in which the yarn may be as S-twist or Z-twist according to the direction of spinning and spun yarn may contain a single type of fiber or be a blend of various types of yarn and possible combining yarn of natural fibers with yarn of synthetic fibers.

The illustrated tanning step may in principle cover a tanning process design and executed on a cord/yarn, where the collagen proteins of the yarn/cord has not previously been tanned. It is a however preferred to regard the tanning step as a re-tanning step if the collagen fiber subjected to the tanning step has previously be subjected to some kind of tanning agent.

It is however noted that the preferred tanning operation is best described as a so-called post-tanning, e.g. involving at least one wet processing step involving a single chemical process or a combination of chemical processes.

It should be noted that the illustrated embodiments of FIG. 4 is exemplary.

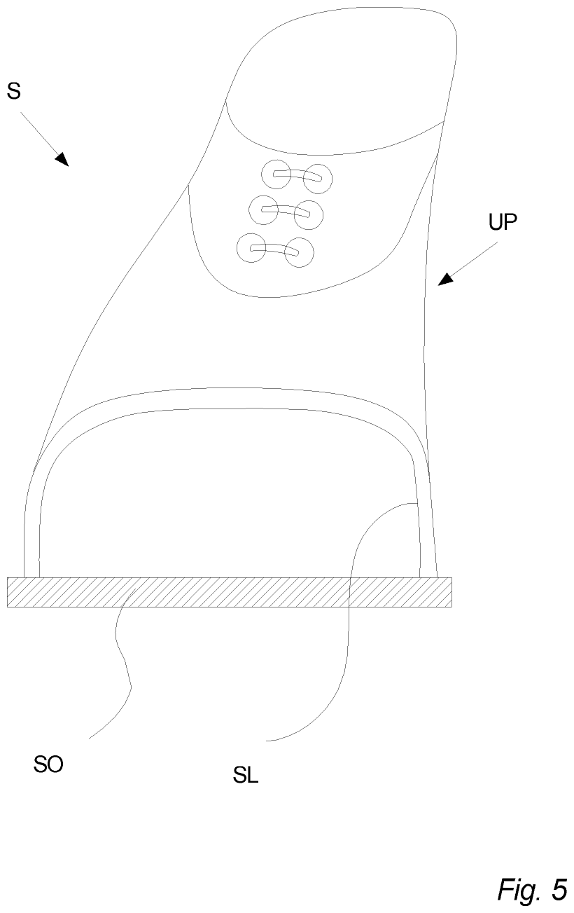

FIG. 5 illustrates a further embodiment of the invention. The embodiment comprises a shoe (S) comprising a sole (SO) and an upper part (UP).

The upper part (UP) may be formed completely in fabric made according to the provisions of the invention. The upper part (UP), may also include a shoe lining (SL).

The shoe lining (SL) is the material inside the shoe that in some applications comes in contact with the entire foot: the sides, top and heels, or at least some of these parts of the foot. A purpose of the lining is to cover the inside seams of the shoe and lengthen the shoe's lifespan. Shoe lining (SL) made according to the present invention may cushion and comfort the foot or draw out moisture.

Thus, the leather fabric lining feels soft on the skin and will conform to the shape of the foot over time. It's also durable and allows air flow, letting moisture evaporate. Although leather is the highest quality material for lining, certain shoes, particularly athletic shoes, don't use leather lining because the leather adds extra weight. The present invention facilitates such use as the leather fabric may provide all or many of the advantages of conventional leather but also be present in a light weight, thereby increasing to application to e.g. athletic shoes, but also by realizing that the incorporated dye may be matched very well to fabric e.g. in terms of amount contrary to conventional leather where such a process is extremely time consuming and difficult to control in terms of desired end-color.

Overall it is noted that the leather fabric provided according to the present in invention is very breathable and that a shoe provided according to the present invention may not only be relatively color-durable but also be used both as out and optional inner layer of a shoe thereby providing a shoe having an upper part which very breathable compared to conventional shoe fabric.

The upper part (UP) may be formed wholly or partly of single layered inventive leather fabric, it may be multilayered including a layer of inventive leather fabric and further optionally comprise a lining (SL).

The single layer of inventive leather fabric may e.g. form part of a vamp, quarter or a tongue of the upper part (UP).

The inventive leather fabric may be oriented towards the outside of the shoe and/or the inside.

If multilayered, the multilayer structure of the upper part may comprise one or more layers of the inventive leather fabric and at least one layer, e.g. a reinforcing layer, comprised of another material.

The multilayered fabric may be laminated together.

If laminated, the multilayered fabric of the upper part (UP) may comprise adhesive bonding a reinforcing fabric to the inventive leather fabric.

The reinforcing fabric may provide strength to the laminate, allowing the leather layer to be thin and the overall laminate to be flexible. The reinforcing fabric is therefore relatively thin and has high tensile strength, high tear strength and low elongation at break.

Suitably, the basis weight for the reinforcing fabric is below 150 g/m², typically below 100 g/m², more typically below 75 g/m², and most typically below 60 g/m².

A suitable method for measuring the basis weight of the reinforcing fabric is ASTM D3776.

Suitably, the ultimate tensile strength (breaking strength) of the reinforcing fabric is above 5 kN/m, more typically above 10 kN/m, or even above 15 kN/m.

The ultimate tensile strength expressed in kN/m is the pulling force required to break a 1 m wide sample of the material. A suitable test for measuring the ultimate tensile strength of the reinforcing fabric is ISO 3376:2011. An alternative test specifically adapted for testing tensile properties of polymer matrix composites which could be used is ASTM D3039.

Suitably, the elongation at break of the reinforcing material (i.e. the elongation of the fabric when stretched to its breaking point) is less than 5%, typically less than 4%, or even less than 3%.

A suitable test for measuring the elongation at break is ISO3376:2011. An alternative test specifically adapted for testing the elongation properties of polymer matrix composites which could be used is ASTM D3039.

Suitably, the tear strength of the reinforcing material is above 25 N, typically above 50 N, or even above 75 N.

A suitable method for measuring the tear strength of the reinforcing material is ISO 3377-1:2011. An alternative test specifically adapted for testing the tear strength of polymer matrix composites which could be used is Mil-C-21189 10.2.4.

It will be clear from the above characteristics that the reinforcing fabric is very low basis weight (and hence typically very thin) yet typically has very high tensile strength and tear strength. Suitable materials that fulfil these requirements include fabrics which include at least one layer comprising high strength fibres.

By “high strength fibre” is meant a fibre having an ultimate tensile strength of above 1500 MPa. A suitable test for measuring the ultimate tensile strength of the fibre is ASTM D3822.

Typical high strength fibres include carbon fibres or high tensile strength polymeric fibres, with suitable high tensile strength polymeric fibres including polyethylene (particularly UHMWPE), polyaramid, polybenzoxazole, and polyaromatic esters.

Suitable high strength fibres that can be used in the reinforcing fabric therefore include carbon fibre, UHMWPE fibres such as Dyneema® available from DSM or Spectra® available from Honeywell; polyaramid fibres such as Kevlar® available from DuPont; polybenzoxazole fibres such as Zylon® available from Toyobo; and polyaromatic esters such as Vectran® available from Kuararay, Inc.

In this context, UHMWPE is “ultra-high molecular weight polyethylene”, which is sometimes also referred to as high-modulus polyethylene (HMPE) or high-performance polyethylene (HPPE). UHMWPE is typically characterized by having an intrinsic velocity of at least 4 dl/g, desirably at least 8 dl/g. Generally, the intrinsic viscosity is less than 50 dl/g, typically less than 40 dl/g.

A suitable methodology for measuring intrinsic viscosity is ASTM D1601-2004 (at 135° C. in decalin, dissolution time 16 hours, with DBPC as an anti-oxidant in an amount of 2 g/l solution, by extrapolating the viscosity as measured at different concentrations to zero concentration).

The at least one layer in the reinforcing fabric comprising the high strength fibres may be woven or nonwoven. However, in order to benefit from the strength properties of the fibres, typically the at least one layer will contain the high strength fibres in an oriented fashion, such as woven (including uniweave), monodirectional or multidirectional fabrics.

Typically, the reinforcing fabric will comprise at least one layer having parallel high strength fibres. Said parallel high strength fibres may optionally be embedded in a resin matrix.

The multilayered fabric including the inventive leather fabric may e.g. form part of a vamp, quarter or a tongue of the upper part (UP).

The inventive leather fabric may be oriented towards the outside of the shoe and/or the inside.

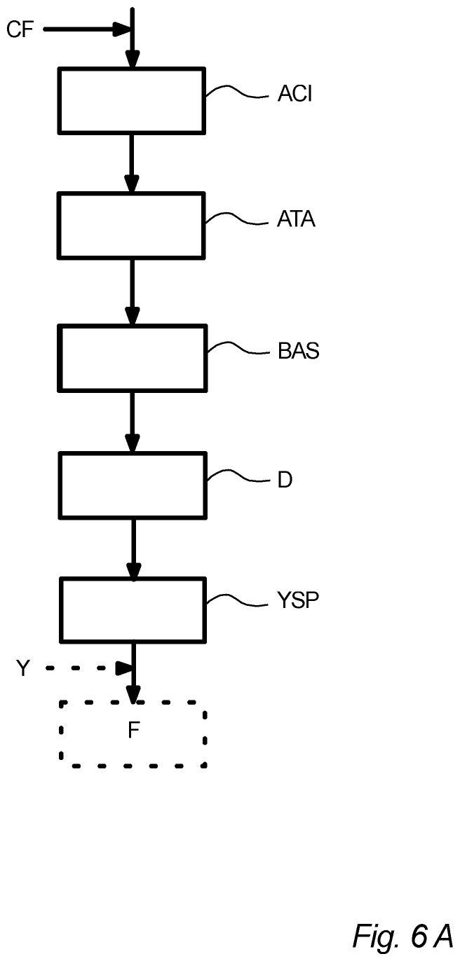

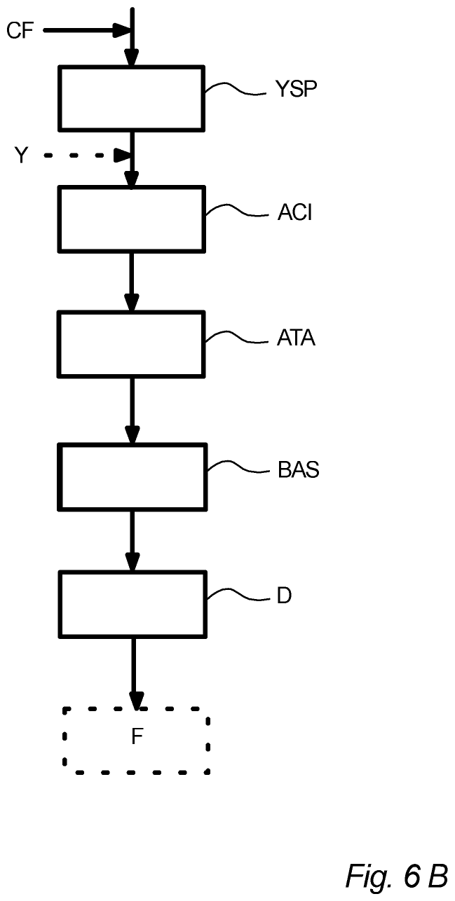

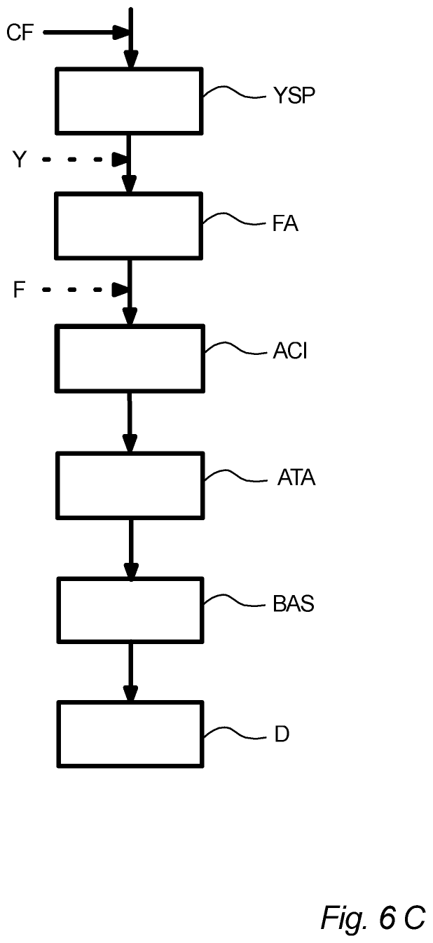

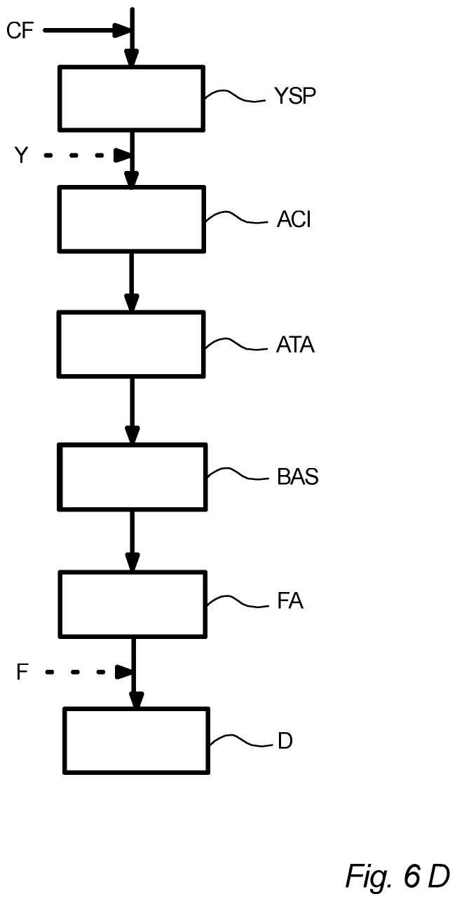

FIG. 6A-6D discloses different ways of applying color within the scope of the invention wherein some embodiments are shown, and others may be applied.

FIG. 6A shows exemplary an embodiment of the invention.

With reference to the drawing, a collagen fiber (CF) is initially subjected to an acidification (ACI) as mentioned above. The collagen fiber (CF) is then subjected to a tanning process/re-tanning (TP) and subsequently subjected to a basification (BAS). The collagen fiber is then subject to a dyeing step (D) resulting in a dyed collagen fiber which is then spun into a yarn (Y) by a yarn spinning process (YSP). The dyed collagen based yarn may then subsequently be processed into a fabric (F).

The present embodiment the illustrates that collagen fibers may be subjected to a tanning process (TP), wherein the collagen fibers are first subjected to acidification and application of tanning agents. This could be a re-tanning and even a first tanning is possible. After application of tanning agents, the collagen fibers are subjected to basification and then to dye, wherein the details related to dyeing are described elsewhere in the description. The dyed fibers are subjected to yarn spinning and possibly manufacturing into fabric. Other processes may be applied in the process, such as i.e. fatliquoring and optional further tanning is possible.

Different variants of the process will be described below. It is however noted that the principle difference in terms of process, not necessarily in terms of the obtained end-product, are referring to the same process steps, just organized in different logic sequences. Only the differences will be highlighted below.

FIG. 6B shows exemplary an embodiment of the invention collagen fibers subjected to a tanning process (TP), wherein the collagen fibers (CF) are first subjected to yarn spinning (YSP). After yarn spinning (YSP) the collagen fibers (CF) are subjected to acidification (ACI), application of tanning agents (TP), basification (BAS) and then dyeing (D). The tanning process (TP) could be a re-tanning and even a first tanning. The dyed collagen fiber-based yarn may optionally be manufactured into fabric (F) and other processes may be applied such as i.e. fatliquoring and optional further tanning is possible.

FIG. 6C shows exemplary an embodiment of the invention collagen fibers (CF) subjected to a tanning process (TP), wherein the collagen fibers (CF) are first subjected to yarn spinning (YSP) and manufacturing into fabric (F). The manufacturing of yarn into fabric is described in detail elsewhere in the description. The fabric is then subsequently subjected to acidification (ACI), application of tanning agents (TP) and basification (BAS) and then dyeing (D).

FIG. 6D shows a further exemplary embodiment corresponding in terms of process steps to the already described process steps of FIG. 2A-C, but now where the collagen fiber (CF) is first spun into a collagen based yarn (Y) by a yarn spinning process (YSP) and where the produced yarn (Y) is subject to tanning agents (TP) and associated acidification (ACI) and basification (BAS) for promotion of the penetration of the tanning agents into the yarn (Y). The yarn (Y) is then subsequently manufactured into a fabric (F) which is subsequently dyed in a dyeing step (D).

The latter embodiment is generally considered a very advantageous embodiment of the invention as the semi-finalized yarn may be manufactured in a durable and distributable state, e.g. in a standard color which is relatively neutral or just a color being a natural result of the tanning agents applied. The standard colored dyed yarn, which is now considered a leather yarn, may then be dyed subsequently, e.g. after manufacturing of the fabric (F). The basic point is that such process has never been attractive in relation to conventional leather not produced by leather yarn, as dyeing is a very complex and demanding step which should typically be performed at the tannery or at least at some kind to the manufacturer, e.g. a shoe manufacturer, requiring the leather in a certain color. This means at the very least that a lot of dyestuff are applied to hides or parts of hides and thereby restricting the use of the applied hide/leather.

The present invention basically facilitates in some embodiments that all yarn may of course be produced in a certain color or produced in one or few color appearances which may subsequently be subject to dyeing into the desired end-color. In other words. The applied dye is applied and fixed for the intended purpose at a very late stage of the process, thereby reducing risks of waste or the manufacturing for storage of leather materials which may be irrelevant at the time they are to be used.

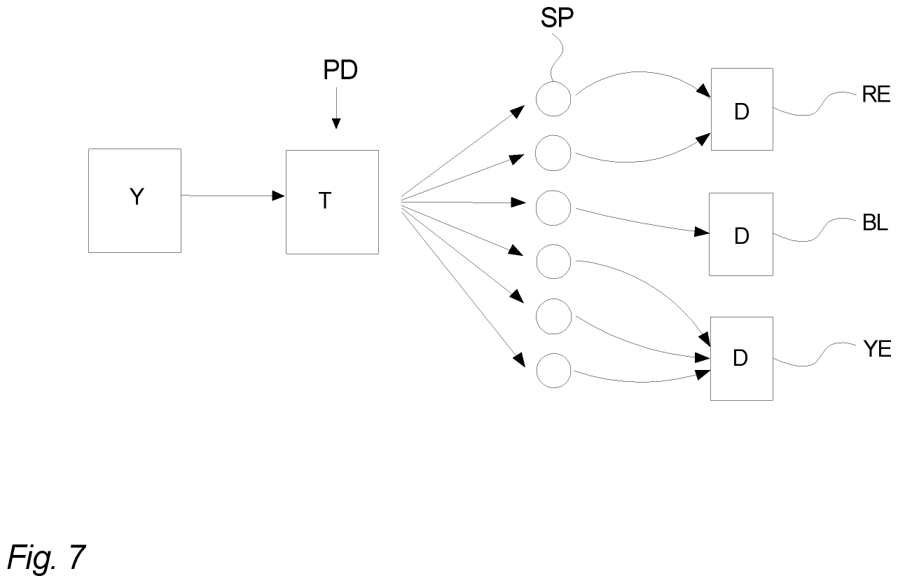

FIG. 7 an example of an embodiment of the invention.

With reference to the drawing, a collagen-based yarn (Y) subjected to a tanning process T. This tanning process may e.g. include or be associated with a dye normalization step e.g. a pre-dyeing (PD)

After the tanning and optional color normalization process, the yarn is wound on reels (SP), or similar suitable constructions for keeping yarn.

Thereafter the yarn reels SP may be picked for subsequent dying into the desired end colors e.g. red (RE), blue (BL) or yellow (YE).

The step of pre-dyeing or normalizing the color of the yarn is an optional step, however, a highly advantageous step wherein the color of the yarn achieves the same neutral base color and thereby optimizing the dyeing (D) process and promotes predictability in relation to the final dyeing process. This is a new a very interesting aspect of the invention as collagen-based products to be dyed, e.g. leather requires complex dyeing, and the results are typically obtained by trial and error, while the present embodiment of the invention makes it possible to perform a much more predictable dyeing, independent of the eventual color of the yarn.

Normalization of the color of the yarn in the present context is generally understood as a process to obtain yarn with the same neutral base color. That may be a discoloring, pre-dying, bleach or other relevant process to obtain a similar neutral base color. In other words, the pre-dyeing may not necessarily include the process of adding dye as bleaching or other chemical processing may be enough to provide the desired neutral base color.

According to an advantageous embodiment of the invention, a so-called neutral base-color may include a color which is able to be dyed into different relevant colors, such as black, white, yellow, red and blue, with basically the same dyeing process, just with colors of dye.

The collagen-based yarn may origin from several sources, e.g. hide such as e.g. bovine, other animals, leather waste or other sources of collagen and the basis color might be different. Different treatments or processes of the hide, waste products etc. may change the color of the products and thereby also the initial color of the collagen-based yarn. Further, the hide may have been treated with tanning agents e.g. chrome tanning, that may provide a blue or grey color of the yarn. Hide treated with vegetable tanning agents may have a more white or yellow appearance. Applying the same amount of dye to yarns with different base colors may become irregular and aesthetic difficult to handle in the final products without applying extra dyeing, dependent on which color the final yarn is intended to have.

It should thus be noted that the present, although very advantageous, providing of a neutral base is extremely attractive both for the purpose of avoiding final leather/collagen-based yarn manufactured in an undesired color. Such a problem is often associated to conventional leather manufacturing. It, on the other hand, also makes it possible to re-user such waste products for the purpose of re-constructing collagen-based fibers, staple fibers, collagen-based yarn even if the waste product starting material has been dyed already or at least partially present in e.g. a bluish color due to an already performed chrome-tanning. In other words, the collagen material applied as the basis for the manufacture of staple fibers/filament/yarn in the present invention, may due to different origin, be inherit very diverse. The term “spool” in this context means a physical object suitable for rolling up yarn for storage or further processes. It may also be a cone, reel or any other suitable object for rolling up yarn. The processing of rolling/winding up the yarn may also simply be performed as to provide a ball of yarn with no physical object onto which the yarn has be wound.

It is noted that the tanning process performed prior to the providing of the yarn on the reels facilitate both subsequent dyeing into a desired end color/use-color but also makes it possible to apply a general yarn material featuring leather properties, which may be utilized and benefitted from in a subsequently produced fabric.

Another advantageous aspect is that the yarn, when reeled up, already is possessing the desired tenacity, strength or elasticity properties required of the produced yarn. In other words, the manufacture of the yarn into a fabric may benefit of the fact that the tanning has already been performed and that the yarn is therefore less susceptible to breakage or that the yarn is at least easier to handle without risking breaking or weakening the yarn during manufacture of the fabric.

### FIGURE REFERENCES

CF. Collagen fiber
Y. Yarn
TY. Tanned yarn
CO. Cord
TE. Tanning equipment
YSP. Yarn spinning process
S. Shoe
UP. Upper part
SO. Sole
SL. Shoe lining
F. Fabric
TP. Tanning process
TF. Tanned fabric
FMP. Fabric manufacturing process.

1. A method of manufacturing a collagen yarn, the method comprising steps of:
providing collagen fiber;
spinning the collagen fiber into a yarn; and
tanning the yarn subsequent to the spinning of the collagen fiber into a yarn.

2. The method according to claim 1, wherein the tanning of the yarn is a re-tanning.

3. The method according to claim 1, wherein the collagen fiber is reconstructed fiber staple fibers produced based on a number of mechanically sub-divided or grinded protein fibrils.

4.-22. (canceled)

23. The method according to claim 1, wherein the tanning of the yarn is a continuous process via rolls pulling the yarn through a tanning compound.

24.-26. (canceled)

27. The method according to claim 1, wherein the collagen yarn comprises at least 40% by weight of collagen protein.

28. The method according to claim 1, wherein the collagen yarn comprises non-collagen protein.

29. The method according to claim 1, wherein the collagen yarn comprises 1 to 60% by weight of non-collagen protein.

30.-39. (canceled)

40. The method according to claim 1, wherein the manufacturing consumes less chemicals than in conventional leather tanning.

41. The method according to claim 40, wherein the manufacturing is performed with chemicals reduced by 10% by weight.

42. The method according to claim 40, wherein the manufacturing is performed with chemicals reduced by 25% by weight.

43.-46. (canceled)

47. The method according to claim 1, wherein a fineness of the yarn is within a range of about 10 Tex to 300 Tex.

48. The method according to claim 2, wherein a content of a tanning agent in the collagen fiber is increased during the re-tanning of the yarn with at least 10% by weight of the tanning agent contained in the yarn previous to the re-tanning.

49.-52. (canceled)

53. The method according to claim 1, further comprising dyeing the yarn,
wherein the yarn is wound up on a plurality of yarn reels prior to dyeing.

54. The method according to claim 1, wherein the collagen fiber has a tenacity of more than 3 g/denier.

55.-57. (canceled)

58. The method according to claim 1, further comprising forming the yarn into a fabric.

59. (canceled)

60. The method according to claim 1, further comprising subjecting the yarn to tanning agents,
wherein the amount of tanning agent is less than 10% by weight.

61. (canceled)

62. A leather shoe comprising:
a sole; and
an upper part,
wherein the upper part is at least partly comprised of leather fabric, and
wherein the leather fabric is produced from leather yarn made according to a method of manufacturing a collagen yarn comprising steps of:
providing collagen fiber;
spinning the collagen fiber into the leather yarn; and
tanning the leather yarn subsequent to the spinning of the collagen fiber into the leather yarn.

63. The leather shoe according to claim 62, wherein the leather fabric is applied in the upper part of the shoe.

64. The leather shoe according to claim 62, wherein the leather fabric is applied as a shoe lining or as a part of a shoe lining.

65.-69. (canceled)

70. The method according to claim 58, wherein the leather yarn of the fabric is provided on basis of a suspension of protein fibrils.
